(19) United States
(12) Patent Application Publication
(10) Pub. No.: US 2023/0183690 A1
(43) Pub. Date: Jun. 15, 2023

(54) EXOSOME DELIVERY OF CANCER THERAPEUTICS

(71) Applicant: MORPHOGENESIS, INC., TAMPA, FL (US)

(72) Inventors: EVREN ALICI, HUDDINGE (SE); MICHAEL J. SHAMBLOTT, ST. PETERSBURG, FL (US); MICHAEL J. P. LAWMAN, TEMPLE TERRACE, FL (US); PATRICIA D. LAWMAN, TEMPLE TERRACE, FL (US)

(21) Appl. No.: 18/055,724

(22) Filed: Nov. 15, 2022

Related U.S. Application Data
(60) Provisional application No. 63/279,218, filed on Nov. 15, 2021.

(51) Int. Cl. C12N 15/11; C12N 15/88; C07K 14/47

(52) U.S. Cl. CPC C12N 15/111; C12N 15/88; C07K 14/4702; C07K 2319/33; C12N 2320/32

(57) ABSTRACT

Chimeric expression constructs encoding an immunogenic polypeptide fused to a lactadherin domain are described. The compositions are useful for delivery of antigens to different cells, including cancer cells.

### CROSS-REFERENCE TO RELATED APPLICATION

This application claims the benefit of U.S. Provisional Application Ser. No. 63/279,218, filed Nov. 15, 2021, the disclosure of which is hereby incorporated by reference in its entirety, including all figures, tables and amino acid and nucleic acid sequences.

### REFERENCE TO SEQUENCE LISTING

The Sequence Listing for this application is labeled “Seq-List.xml” which was created on Nov. 15, 2022 and is 23,828 bytes. The entire content of the sequence listing is incorporated herein by reference in its entirety.

### FIELD OF THE INVENTION

The present invention relates to delivery of mRNA encoding selected antigens to specific cancer cells. In particular, mRNA coding for Emm55, an immunogenic protein, can be delivered using extracellular vesicles.

### BACKGROUND OF THE INVENTION

Some of the limitations faced by the current generation of emm55-based vaccines are that they rely on inefficient and untargeted transfection methods to impart Emm55 protein expression. For example, injection of naked DNA or nucleic acids complexed to synthetic cationic lipid-based delivery reagents. Although effective, this approach limits the vaccine to easily accessible tumors, and to tumors that readily take up and express exogenous nucleic Exosomes, sometimes referred to as small extracellular vesicles (EV), are 30- to 150-nm diameter membrane vesicles secreted into the extracellular space by virtually all cells, including tumor cells. They bind or fuse to the plasma membrane of neighboring cells in a process thought to allow cell-to-cell communication, antigen presentation, protein secretion, and shuttling of cell surface, cytosolic and nuclear proteins, lipids, DNA, mRNA and non-coding RNA.

Exosomes can be isolated by ultracentrifugation, precipitation with polymers such as polyethylene glycol, size exclusion chromatography, ultrafiltration or tangential flow filtration, or immunoaffinity chromatography. Once isolated, exosomes can be cryopre served with dimethyl sulfoxide or sugars such as trehalose, and can be freeze dried to further extend storage (Zhang et. al. doi:10.2147/IJN.S264498).

Due to size and composition, exosomes can avoid phagocytosis by macrophages and can readily cross blood vessel walls, extracellular matrices, blood-brain barrier and other biological barriers. The presence of CD55 and CD59 on their surface allows exosomes to avoid opsonin activation and coagulation. This enhances bioavailability compared to similarly sized synthetic structures such as liposomes.

One of the most important properties of exosomes is that they can be readily targeted to intended recipient cells. Natural cell-produced exosomes can recognize specific cell types via their surface receptors. For example, exosomes with Tspan8 preferentially bind to CD11b and CD54-positive cells (Rana et. al. doi: 10.1016/j.biocel.2012.06.018). Further targeting can be achieved by engineering of donor cells to express a targeting protein fused to an exosomal membrane protein such as lysosome-associated membrane glycoprotein 2b (Lamp2b) or the tetraspanins CD63 and CD9 (Stickney et. al. doi: 10.1016/j.bbrc.2016.02.058).

Exosomes are of interest as a delivery mechanism for vaccines as they can provide immune-system activating signals through several different mechanisms. For example, exosomes produced by antigen-presenting cells (APC) contain MHC class I and class II complexes along with costimulatory and adhesion proteins. Purified exosomes can activate CD4 or CD8 positive T cells, as well as hematopoietic stem cells, B cells and NK cells. Furthermore, exosomes purified from other cell types can spread antigens or peptide-loaded MHC complexes to APCs for more efficient presentation. Exosomes derived from tumor cells can contain tumor associated antigens.

One modality used for exosome display of antigens is the generation of chimeric expression constructs encoding a protein of interest fused to the C1C2 domain of lactadherin. Lactadherin is released via the exosome secretory pathway by binding to the vesicle surface, and chimeric proteins containing the C1C2 domain of lactadherin have been shown to be released into the extracellular milieu bound to exosomes. Exosomes can also be engineered to traverse the blood brain barrier and target specific, and difficult to transfect cell types.

Cancer vaccines using the Streptococcus pyogenes emm55 gene have been developed and tested extensively in companion animal cancer patients and in a clinical trial setting for the treatment of melanoma in horses. These vaccines involve production of Emm55 protein from the emm55 gene itself or from emm55 mRNA (SEQ ID NO: 1). Once expressed on tumor cells, Emm55 protein is thought to stimulate the host immune system, and thereby help augment inflammatory anti-tumor antigen response to provide broad anti-tumor immunity.

There is a naturally occurring precedent for combining emm55 expression with exosome delivery. Mycobacterium bovis BCG-infected macrophages release exosomes containing mycobacterial antigen that, in the presence of dendritic cells, promote T-cell immunity in mice. Similarly, exosomes can contain immune stimulatory viral antigens.

U.S. Pat. No. 9,636,388 describes transformation of cancer cells with mRNA encoding the immunogenic protein Emm55. Cells into which mRNA was introduced expressed the immunogenic protein. The corresponding DNA also expressed the same immunogenic protein in lymphoma cells and was demonstrated to be useful as an in vivo vaccine.

### SUMMARY OF THE INVENTION

Some of the limitations faced by the current generation of emm55-based vaccines include reliance on inefficient and untargeted transfection methods to impart Emm55 protein expression. For example, injection of naked DNA or nucleic acids complexed to synthetic cationic lipid-based delivery reagents, although effective, limits the vaccine to easily accessible tumors, and to tumors that readily take up and express exogenous nucleic acids.

Delivery of emm55 to tumors by exosomes offers several advantages that may circumvent these limitations and provide enhanced anti-cancer immunity, including:
(1) Increased transfection efficiency and expression of Emm55 by cell types that are accessible (through direct injection) and capable of transfection using cationic lipid-based reagents.
(2) Increased transfection efficiency of a fusion protein consisting of the emm55 gene and the Lactadherin C1C2 domain, or other protein domain capable of targeting a protein to an exosome, by cell types that are accessible and capable of transfection using cationic lipid-based reagents.
(3) Increased transfection efficiency of emm55 and expression of Emm55 by cell types that are accessible but refractory to transfection using cationic lipid-based reagents.
(4) Increased transfection efficiency of a fusion protein consisting of the emm55 gene and the Lactadherin C1C2 domain, or other protein domain capable of targeting a protein to an exosome, by cell types that are accessible but refractory to transfection using cationic lipid-based reagents.
(5) Increased transfection efficiency of emm55 and expression of Emm55 by cell types that are not accessible by injection (such as lymphoma cells).
(6) Increased transfection efficiency of a fusion protein consisting of the emm55 gene and the Lactadherin C1C2 domain, or other protein domain capable of targeting a protein to an exosome, by cell types that are not accessible by injection.
(7) Targeting Emm55 protein expression to a specific cell type based on inclusion of specific proteins on the exosome surface.
(8) Targeting Emm55 protein expression to the central nervous system by crossing the blood brain barrier based on inclusion of specific proteins on the exosome surface.
(9) Enhanced anti-cancer immune response by inclusion of nucleic acids coding for immune costimulatory molecules.
(10) Enhanced anti-cancer immune response by derivation of exosomes from dendritic cells or other antigen presenting cells loaded with Emm55 peptide(s).
(11) Enhanced anti-cancer immune response by inclusion of siRNAs or other non-coding RNAs that inhibit immunosuppressive signaling.
(12) Enhanced anti-cancer immune response by derivation of exosomes from purposefully MHC-mismatched cells.

### BRIEF DESCRIPTION OF THE DRAWINGS

FIG. 1. Western blot analysis of Emm55 expression by stably transduced HEK293FT cells. Antibody binding to actin was used as a loading control. LeGo-iG2/empty is protein lysate from HEK293FT cells transduced with LeGo-iG2 lentiviral vector lacking emm55 sequence.

FIG. 2. Subcellular localization of Emm55 in RPMI-8226 that were transduced with LeGO-iG2-emm55-JCat and LeGO-iG2-empty lentivirus.

FIG. 3. Nanoparticle Tracking Analysis (NTA) measurements. Extracellular Vesicle (EV) characterization in HEK293FT cells stably expressing emm55. Average size of EVs produced from HEK293FT cells transfected with LeGO-iG2-emm55-JCat.

FIG. 4. Western blot analysis of Emm55 protein expression in Extracellular Vesicles (EVs) from HEK293FT cells with LeGO-iG2-emm55-JCat and LeGO-iG2-emm55-MF.

FIG. 5. Agarose gel electrophoresis detection of emm55 mRNA PCR amplimers from RNA isolated from RPMI-8226 cells and EVs.

### EXAMPLES

### Example 1. Emm55-Containing Exosome Used to Improve Transfection Efficiency of Tumor Cells In Vitro

Murine B16 cell lines are created by stable transfection with a mammalian expression construct encoding either emm55, a C-terminal fusion protein consisting of emm55 and the Lactadherin C1C2 domain, Lactadherin C1C2 domain only (SEQ ID NO:5), or expression vector only (empty vector). Exosomes obtained from cell lines are evaluated for the level of emm55 expression by western blot and quantitative RTPCR. Exosomes containing the highest level of emm55 mRNA or protein are used to transfect the murine melanoma B16 cell line in vitro then screened for Emm55 protein expression compared to negative controls.

### Example 2. Emm55-Containing Exosome Used to Improve Transfection Efficiency of Tumor Cells In Vivo

Exosomes derived from B16 cell lines expressing either emm55 mRNA or a C-terminal fusion protein consisting of emm55 and the Lactadherin C1C2 domain are injected intratumorally into mice that have been transplanted with B16 cells. Tumor burden, animal survival and tumor emm55 expression of emm55 exosome-injected mice are compared to mice injected with negative control exosomes.

### Example 3. Emm55-Containing Exosome Used to Transfect Peripheral Blood Mononuclear Cells (PBMCs) In Vitro

Resting PBMCs are refractory to transfection by electroporation or cationic lipid-based reagents. Human PBMC-derived exosomes containing emm55, a C-terminal fusion protein consisting of emm55 and the Lactadherin C1C2 domain, or negative controls. After 24 and 48 hours of culture, PBMCs are harvested to determine emm55 mRNA and protein expression levels.

### Example 4. Emm55-Containing Exosome Used to Transfect Murine B-Cell Lymphoma In Vitro and In Vivo

The Eμ-myc murine model can be used to study the progression of spontaneous lymphoma from follicular to diffuse large B-cell lymphoma (DLBCL). Harvested lymphoma cells are treated with exosomes containing emm55, a C-terminal fusion protein consisting of emm55 and the Lactadherin C1C2 domain, or negative controls in vitro. An emm55 mRNA and protein expression time course is then established. Mice are then transfused with ex vivo exosome-transfected cells or lymphoma-derived exosomes and survival is compared to negative controls.

### Lentiviral Transduction of EV-Producing Cell Lines

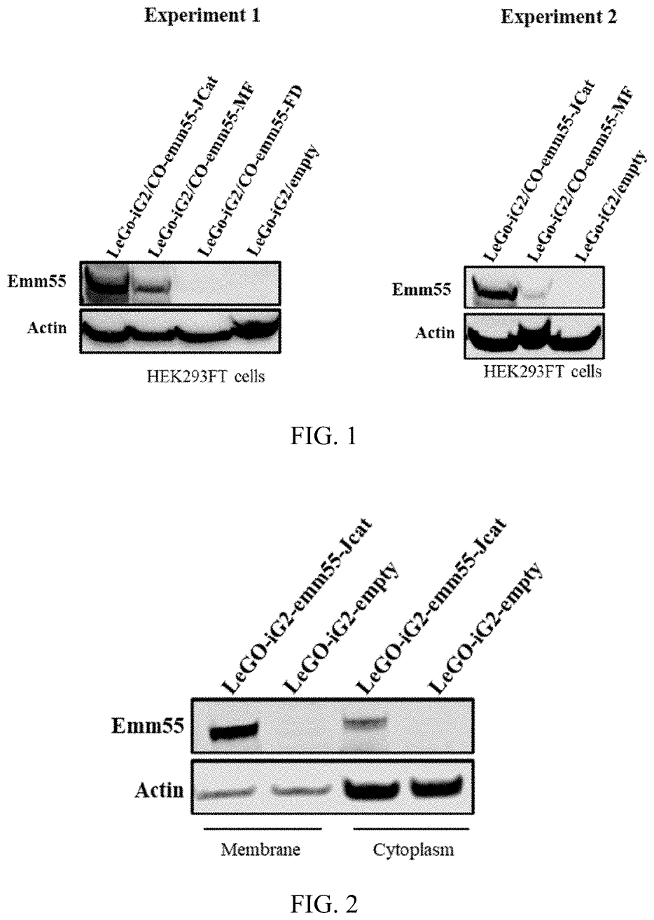

To improve protein translation efficiency, the coding region of emm55 was modified to replace codons that are inefficiently translated in mammalian cells. Optimization was carried out using 3 algorithms and all three resultant emm55 sequences were cloned into the LeGO-iG2 lentiviral expression vector upstream of the internal ribosome binding site (IRES), such that both Emm55 and enhanced green fluorescent protein (eGFP) would be translated from a single mRNA. The three emm55 sequence variants (SEQ ID NO:2, SEQ ID NO:3 and SEQ ID NO:4) are named JCat, MF and FD, after the algorithms which produced the optimized sequence. The three lentiviral constructs were used to transduce HEK293FT and RPMI-8226 cell lines, both of which have been use to isolate EVs. Expression of Emm55 was evaluated using western blots stained with chicken anti-Emm55, an antibody developed by Morphogenesis. In two independent experiments, the emm55-JCat variant produced the highest level of Emm55 protein (FIG. 1).

Protein lysates from RPMI-8226 cells transfected with the LeGO-iG2-emm55-JCat and LeGO-iG2-empty vectors were also evaluated by western blotting. In this experiment, protein lysates were prepared using reagents that differentially extract proteins from the cell membrane and cytoplasm (ThermoScientific Catalog #89842). As seen in FIG. 2, Emm55 is detected in both compartments.

From the data presented in FIGS. 1 and 2, it can be concluded that both HEK293FT and RPMI-8226 cells were successfully transduced with the LeGO-iG2 lentivirus.

### EV Production by HEK293FT and RPMI-8226 Cells

EVs were harvested and purified from cell cultures using the following method.

1. Expanded RPMI-emm55-JCat and RPMI-mock-JCat cells cultured in T175 flasks containing 7E5 cells/ml with 50 ml fresh complete medium (RPMI+10% FBS) for 24 hours.

2. Cells were collected into 50 ml tubes, centrifuged at 350×g for 5 minutes and the complete medium was replaced with Opti-MEM containing 1E6 cells/ml.

3. After 48 hours the media were collected and centrifuged at 700×g for 5 minutes then at 2000×g for 10 minutes.

4. The supernatant was filtered through 0.22 μm filter.

5. EVs were purified on (mPES) hollow fiber filters with 300 kDa membrane pore size (MidiKros, 370 cm2 surface area, SpectrumLabs) in PBS.

6. An Amicon Ultra-15, molecular weight cut-off spin-filter (Millipore) was used to concentrate the samples to a final volume of 200-500 μL.

7. Nanosight Tracking Analysis (NTA) was used to measure the concentration and size of EVs.

RNA and cDNA were prepared from EVs using the following method.

1. 300 μl TRIzol reagent (ThermofisherScientific) was added to For 1E11 EVs collected in a microfuge tube and vortexed and incubated at room temperature (RT) for 5′.

2. 100 μl chloroform was added.

3. After incubation for 5 minutes at RT, tubes were centrifugated 12,000×g, 4° C., 15 minutes.

4. Aqueous solution was taken into a new Eppendorf tube. 5. 2 μl of glycol blue dye was added and incubated at RT for 10 minutes.

6. The tubes were centrifuged at 12000×g, 7° C., 10 minutes and supernatant was removed.

7. Pellet was washed 2 times with 200 μl of 75% ethanol and centrifugated 9,000×g for 10 minutes.

8. The aqueous part was discarded and the pellet was dried 10 minutes in RT.

The pellet was resuspended in 25 μl RNAse free water.

9. cDNA was synthesized from 1 μg RNA using the iScript Reverse transcription kit (Bio-rad).

10. PCR was carried out on cDNA using emm55-JCat specific primers using 35 cycles of 95° C., 1 min., 55° C., 1 min., 72° C., 1.5 min. followed by a final extension at 72° C. for 7 min. The primers were as follows:

JCat Primer 5'-453 bp
(SEQ ID NO: 6)
Fp1: AAAAGCAAGTTCCAGGAC
(SEQ ID NO: 7)
Rp1: CTCCTTCTTCACCAGCTC
JCat Primer Middle-402 bp
(SEQ ID NO: 8)
Fp2: AGAACAAGAAGGAGGAGC
(SEQ ID NO: 9)
Rp2: TGTACTGGCTCATGAAGG
JCat Primer 3'-524 bp
(SEQ ID NO: 10)
Fp3: TGGAGGAGCAGAACAAGA
(SEQ ID NO: 11)
Rp3: CTTGGTCTCCTTCATGGG

EV uptake was determined using the MM.1S multiple myeloma cell line. Briefly, 1×106 MM.1S cells were seeded in 6 well plate with 2 ml DMEM medium supplemented with 10% FBS and 1×1011 Emm55 EVs were added. After 10 hours incubation with EVs, the cells were washed and lysed according to WB protocol for detection of Emm55 protein in the recipient cells.

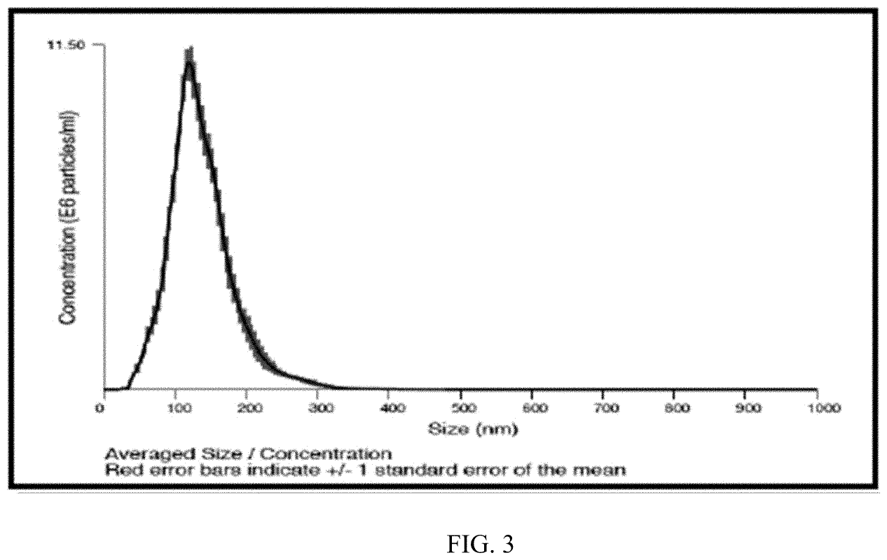

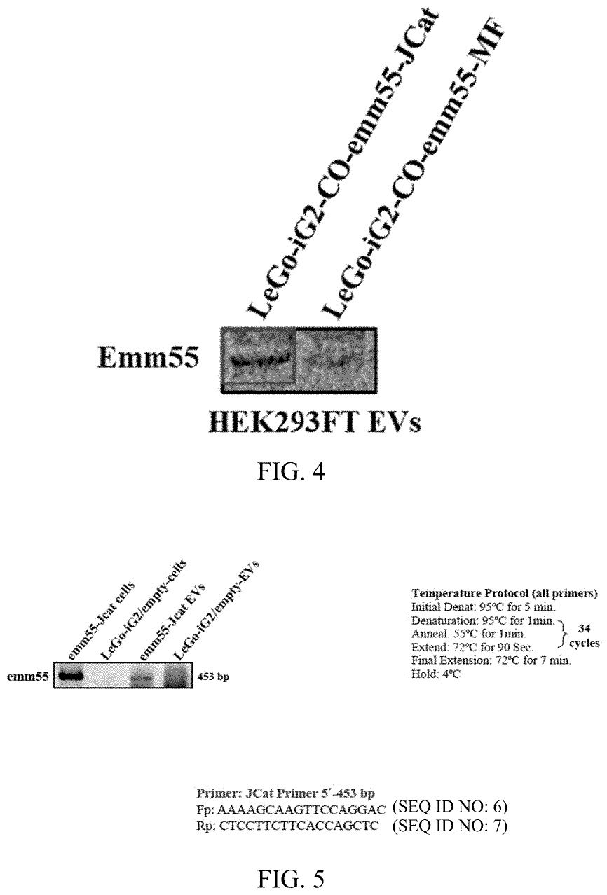

Nanosight tracking analysis (NTA) was used to measure the concentration and sizes of the EVs isolated from HEK293FT cells transfected with LeGO-iG2/CO-emm55-JCat. The mean size distribution of the EVs from HEK293FT cells transfected with LeGO-iG2/CO-emm55-JCat was 135+/−1 nm (FIG. 3).

Emm55 mRNA expression was also detected in RPMI-8226 cells and EVs (FIG. 5).

We claim:

1. An exosome composition comprising a plasmid consisting of a nucleic acid encoding an immunogenic protein fused with a lactadherin C1C2 domain.

2. The exosome composition of claim 1 wherein the immunogenic protein is a streptococcal antigen encoded by the nucleic sequence of SEQ ID NO:1.

3. The exosome composition of claim 1 wherein the immunogenic protein is encoded by a nucleic acid selected from SEQ ID NO:2, SEQ ID NO:3 and SEQ ID NO:4.

4. The exosome composition of claim 1 wherein the lactadherin C1C2 domain is encoded by a nucleic acid having the sequence of SEQ ID NO:5.

5. The exosome composition of claim 1 wherein the plasmid is a lentiviral expression vector consisting of Emm55 DNA and lactadherin C1C2 domain DNA.

6. An exosome composition comprising human peripheral blood mononuclear cells transfected with an mRNA or DNA of claim 1.

7. A method of delivering an immunogenic protein to a cell, comprising administering the exosome composition of claim 1 to a cancer cell.

8. The method of claim 7 wherein the cancer cell is a lymphoma, peripheral blood mononuclear, dendritic cell or cancer cell.

9. The method of claim 8 wherein the cancer cells are transfected ex vivo.

10. The method of claim 7 wherein the immunogenic protein has the sequence of SEQ ID NO:1.

11. The method of claim 9 wherein the exosome isolated from the transfected cancer cells is administered to said subject.

12. A method of treating cancer comprising obtaining beta-cell lymphoma cells transfected ex vivo with exosomes containing the expression construct of claim 5 and administering the transfected exosomes to a lymphoma cancer subject in need thereof.


SEQUENCE LISTING (11 sequences)

SEQ ID NO: 1 - RNA, 1854 nt, synthetic construct
  misc_feature 1..1854: note = Theoretical emm55 mRNA sequence
  misc_feature 1..2: note = 5' ARCA methylguanosine cap
  misc_feature 2..132: note = 5' untranslated region
  misc_feature 133..135: note = start codon
  misc_feature 133..1804: note = emm55 gene
  misc_feature 1806..1854: note = Poly(A) tail, minimum of 50 adenosines
  source 1..1854: mol_type = other RNA
gggagaagatctttgtcgatcctaccatccactcgacacacccgccagcggccgctgccaagcttccgagctcaagcttcgaattctgcagtcgacggtaccgcgggcccgggatccataaggagcataaaaatggctaaaaataccacgaatagacactattcgcttagaaaattaaaaacaggaacggcttcagtagcagtagctttgactgttttagggacaggactggtagcagggcagacagtaaaagcaagccaaacagaaccatctcagaccaataacagattatatcaagaaagacaacgtttacaggatttaaaaagtaagtttcaagacctgaaaaatcgttcagagggatacattcagcaatactacgacgaagaaaagaacagtggaagtaactctaactggtacgcaacctacttaaaagaattaaatgacgaatttgaacaagcttataatgaacttagtggtgatggtgtaaaaaaattagctgcaagtttgatggaagaaagagtcgctttaagagacgaaatcgatcagattaagaaaatatcagaagaattaaaaaataagctgagagcaaaagaagaagaattaaaaaataaaaaagaggaacgtgagcttgagcatgctgcctatgcagcagatgcaaagaaacatgaagaatatgtcaaatccatgtctctcgtactaatggataaagaagaggagcgtcataaactagagcaatcattagacacggctaaagctgagcttgttaaaaaagagcaagagttacagttagtcaaaggcaatctagatcaaaaagaaaaagaactagaaaatgaagagctagcgaaagaaagtgctattagtgatttgactgagcagattactgctaagaaggctgaagtagaaaaattaactcaagatttagctgctaagtctgctgaaattcaggaaaaagaagctgaaaaagatcgccaacagcatatgtacgaagcgtttatgagccagtacaaagaaaaagttgagaaacaagagcaagagcttgctaagctaaaacaacttgaaaccatcaacaacaatctattaggtaatgctaaggatatgatagctaagttgtctgctgaaaatgaacaattagcaagcgacaaagcaaaacttgaagaacaaaacaagatttcagaagcgagccgtaaaggtcttcgtcgtgacttggacgcatcacgtgaagctaagaaacaagttgaaaaagatttagcaaacttgactgctgaacttgataaggttaaagaagataaacaaatttcagacgcaagccgtaaaggtcttcgtcgtgacttggacgcatcacgtgaagctaagaaacaagttgaaaaagctttagaagaagcaaacagcaaattagcggctcttgaaaaacttaacaaagagcttgaagaaagcaagaaattaacagaaaaagaaaaagctgagctacaagcgaaacttgaagcagaagcaaaagcactcaaagaacaattagcgaaacaagctgaagaacttgcaaaactaagagctggaaaagcatcagactcacaaacccctgatgcaaaaccaggaaacaaagttgttccaggtacaggtcaagcaccacaagcaggcacaaaacctaaccaaaacaaagcaccaatgaaggaaactaagagacagttaccatcaacaggtgaagcagctaatccattctttacagcggcagcccttactgttatggcaacagctggagtagcagcagttgtaaaacgcaaagaagaaaacgaagctgaattctgcagataaaaaaaaaaaaaaaaaaaaaaaaaaaaaaaaaaaaaaaaaaaaaaaaaa

SEQ ID NO: 2 - DNA, 1707 bp, synthetic construct
  misc_feature 1..1707: note = Synthetic
  source 1..1707: mol_type = other DNA
gccaccatggccaagaacaccaccaaccgccactacagcctgcgcaagctcaagaccggcaccgccagcgtggccgtggccctgaccgtgctgggcaccggcctggtggccggccagaccgtgaaggccagccagaccgagcccagccagaccaacaaccgcctgtaccaggagcgccagcgcctgcaggacctgaaaagcaagttccaggacctgaagaaccgcagcgagggctacatccagcagtactacgacgaggagaagaacagcggcagcaacagcaactggtacgccacctacctgaaggagctgaacgacgagttcgagcaggcctacaacgagctgagcggcgacggcgtgaagaagctggccgccagcctgatggaggagcgcgtggccctgcgcgacgagatcgaccagatcaagaagatcagcgaggagctgaagaacaagctgcgcgccaaggaggaggagctgaagaacaagaaggaggagcgcgagctggagcacgccgcctacgccgccgacgccaagaagcacgaggagtacgtgaaaagcatgagcctggtgctgatggacaaggaggaggagcgccacaagctggagcagagcctggacaccgccaaggccgagctggtgaagaaggagcaggagctgcagctggtgaagggcaacctggaccagaaggagaaggagctggagaacgaggagctggccaaggagagcgccatcagcgacctgaccgagcagatcaccgccaagaaggccgaggtggagaagctgacccaggacctggccgccaagagcgccgagatccaggagaaggaggccgagaaggaccgccagcagcacatgtacgaggccttcatgagccagtacaaggagaaggtggagaagcaggagcaggagctggccaagctgaagcagctggagaccatcaacaacaacctgctgggcaacgccaaggacatgatcgccaagctgagcgccgagaacgagcagctggccagcgacaaggccaagctggaggagcagaacaagatcagcgaggccagccgcaagggcctgcgccgcgacctggacgccagccgcgaggccaagaagcaggtggagaaggacctggccaacctgaccgccgagctggacaaggtgaaggaggacaagcagatcagcgacgccagccgcaagggcctgcgccgcgacctggacgccagccgcgaggccaagaagcaggtggagaaggccctggaggaggccaacagcaagctggccgccctggagaagctgaacaaggagctggaggagagcaagaagctgaccgagaaggagaaggccgagctgcaggccaagctggaggccgaggccaaggccctgaaggagcagctggccaagcaggccgaggagctggccaagctgcgcgccggcaaggccagcgacagccagacccccgacgccaagcccggcaacaaggtggtgcccggcaccggccaggccccccaggccggcaccaagcccaaccagaacaaggcccccatgaaggagaccaagcgccagctgcccagcaccggcgaggccgccaaccccttcttcaccgccgccgccctgaccgtgatggccaccgccggcgtggccgccgtggtgaagcgcaaggaggagaacgaggccgagttctgccgctaccccagccactggcgcccccgcctgtaa

SEQ ID NO: 3 - DNA, 1707 bp, synthetic construct
  misc_feature 1..1707: note = Synthetic
  source 1..1707: mol_type = other DNA
gccaccatggccaagaacaccaccaacagacactacagcctgagaaagctcaagaccggcaccgccagcgtggccgtggccctgaccgtgctgggcaccggcctggtggccggccagaccgtgaaggccagccagaccgagcccagccagaccaacaacagactgtaccaggagagacagagactgcaggacctgaaaagcaagttccaggacctgaagaacagaagcgagggctacatccagcagtactacgacgaggagaagaacagcggcagcaacagcaactggtacgccacctacctgaaggagctgaacgacgagttcgagcaggcctacaacgagctgagcggcgacggcgtgaagaagctggccgccagcctgatggaggagagagtggccctgagagacgagatcgaccagatcaagaagatcagcgaggagctgaagaacaagctgagagccaaggaggaggagctgaagaacaagaaggaggagagagagctggagcacgccgcctacgccgccgacgccaagaagcacgaggagtacgtgaaaagcatgagcctggtgctgatggacaaggaggaggagagacacaagctggagcagagcctggacaccgccaaggccgagctggtgaagaaggagcaggagctgcagctggtgaagggcaacctggaccagaaggagaaggagctggagaacgaggagctggccaaggagagcgccatcagcgacctgaccgagcagatcaccgccaagaaggccgaggtggagaagctgacccaggacctggccgccaagagcgccgagatccaggagaaggaggccgagaaggacagacagcagcacatgtacgaggccttcatgagccagtacaaggagaaggtggagaagcaggagcaggagctggccaagctgaagcagctggagaccatcaacaacaacctgctgggcaacgccaaggacatgatcgccaagctgagcgccgagaacgagcagctggccagcgacaaggccaagctggaggagcagaacaagatcagcgaggccagcagaaagggcctgagaagagacctggacgccagcagagaggccaagaagcaggtggagaaggacctggccaacctgaccgccgagctggacaaggtgaaggaggacaagcagatcagcgacgccagcagaaagggcctgagaagagacctggacgccagcagagaggccaagaagcaggtggagaaggccctggaggaggccaacagcaagctggccgccctggagaagctgaacaaggagctggaggagagcaagaagctgaccgagaaggagaaggccgagctgcaggccaagctggaggccgaggccaaggccctgaaggagcagctggccaagcaggccgaggagctggccaagctgagagccggcaaggccagcgacagccagacccccgacgccaagcccggcaacaaggtggtgcccggcaccggccaggccccccaggccggcaccaagcccaaccagaacaaggcccccatgaaggagaccaagagacagctgcccagcaccggcgaggccgccaaccccttcttcaccgccgccgccctgaccgtgatggccaccgccggcgtggccgccgtggtgaagagaaaggaggagaacgaggccgagttctgcagataccccagccactggagacccagactgtaa

SEQ ID NO: 4 - DNA, 1707 bp, synthetic construct
  misc_feature 1..1707: note = Synthetic
  source 1..1707: mol_type = other DNA
gccaccatggcaaagaataccacgaatagacattattcattaagaaagctcaagaccggcacggcatccgtggcagtggctttgacagtgctgggcacaggcctcgtggccggccagacagttaaggccagccagacagagccatcacaaacgaataaccggctgtaccaggagcggcaaagactgcaggacttaaagtctaaatttcaggatcttaaaaaccggagtgaaggttatatccagcagtattatgacgaggagaagaactccgggagcaatagcaactggtacgcaacctatctgaaggaacttaatgacgagtttgaacaggcttataatgaactgtccggtgatggagtaaagaaattggccgctagtctcatggaagagcgagtggctttgcgggatgagattgaccagattaagaaaatctccgaagagctgaaaaataaacttagggctaaggaggaggagctcaaaaataaaaaggaagaaagagaactggagcacgccgcgtacgccgctgatgctaaaaaacatgaggagtatgtcaagagcatgtccctggtcctgatggacaaggaggaagagcggcacaagctcgagcagtcactggataccgcgaaagcggaactggtgaagaaagaacaagaactccaactagtgaaaggcaatttagaccagaaggagaaggaactagaaaatgaagagctggcgaaagagtctgccatatcagacttgacagagcagatcactgctaagaaggccgaagtggaaaagttgacccaagacttagcagctaaatcggccgagattcaagagaaggaggctgagaaggaccgccagcagcacatgtacgaagcttttatgtcacagtacaaagagaaagtcgagaaacaggagcaggaactggccaaactgaagcagctggagacaataaataataatctactgggcaacgctaaggatatgatcgctaaactgagcgctgaaaacgagcagttagcctccgataaagcgaaattggaagagcaaaacaagatctccgaggccagcaggaaaggccttagaagggacctagacgcctcgagagaggccaaaaagcaggtcgagaaggacctagcaaaccttaccgccgagctggacaaagttaaggaggataagcagatcagcgacgccagtagaaaaggcttaaggagggacttggacgctagtagagaggccaaaaagcaggtggaaaaagcgttagaggaggcgaattccaaattagcggcattggagaagctgaacaaggagctggaggagtcgaagaagttaacggagaaggaaaaagcagagttgcaagcaaagttggaggccgaggcaaaggcattaaaagaacagctcgctaaacaggcggaggaactggcaaaactgagagcaggcaaggcctccgacagtcagacgcctgatgcaaagcctgggaataaggtggtgcctggcacggggcaggccccccaggctggcacgaagccaaatcaaaacaaggctccaatgaaggagactaagcgccagctgcctagtaccggagaggccgccaatccttttttcaccgccgccgcccttactgttatggccactgctggagttgcggctgtggtgaaacggaaagaggaaaacgaagccgaattttgccggtaccctagtcattggagaccaagattgtaa

SEQ ID NO: 5 - protein, 395 aa, synthetic construct
  REGION 1..395: note = hLACTLF/His
MPRPRLLAALCGALLCAPSLLVALDICSKNPCHNGGLCEEISQEVRGDVFPSYTCTCLKGYAGNHCETKCVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGCTGHHHHHH

SEQ ID NO: 6 - DNA, 18 bp, synthetic construct
  misc_feature 1..18: note = primer
  source 1..18: mol_type = other DNA
aaaagcaagttccaggac

SEQ ID NO: 7 - DNA, 18 bp, synthetic construct
  misc_feature 1..18: note = primer
  source 1..18: mol_type = other DNA
ctccttcttcaccagctc

SEQ ID NO: 8 - DNA, 18 bp, synthetic construct
  misc_feature 1..18: note = primer
  source 1..18: mol_type = other DNA
agaacaagaaggaggagc

SEQ ID NO: 9 - DNA, 18 bp, synthetic construct
  misc_feature 1..18: note = primer
  source 1..18: mol_type = other DNA
tgtactggctcatgaagg

SEQ ID NO: 10 - DNA, 18 bp, synthetic construct
  misc_feature 1..18: note = primer
  source 1..18: mol_type = other DNA
tggaggagcagaacaaga

SEQ ID NO: 11 - DNA, 18 bp, synthetic construct
  misc_feature 1..18: note = primer
  source 1..18: mol_type = other DNA
cttggtctccttcatggg